(19) United States
(12) Patent Application Publication
(10) Pub. No.: US 2006/0210976 A1
(43) Pub. Date: Sep. 21, 2006

(54) Methods and nucleic acids for the analysis of methylation patterns within the dd3 gene

(75) Inventor: Torsten Horns, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: EPIGENOMICS AG, BERLIN (DE)

(21) Appl. No.: 10/520,992

(22) PCT Filed: Jun. 25, 2003
(86) PCT No.: PCT/EP03/06690

§ 371 Date: Dec. 22, 2005

(30) Foreign Application Priority Data
Jul. 8, 2002 (EP) 102 30 692.3

(51) Int. Cl. C12Q 1/68; C07H 21/04

(52) U.S. Cl. 435/6; 536/24.3

(57) ABSTRACT

The disclosed invention provides methods and sequences for the analysis of methylation patterns within the 5′ and promoter regions of the gene DD3.

### PRIOR ART

The gene DD3, also known as PCA3, is located at 9q21-22 and its sequence is publicly available at GenBank (Accession number AF103907). The structure of the gene has previously been described (Bussemakers M J, van Bokhoven A, Verhaegh G W, Smit F P, Karthaus H F, Schalken J A, Debruyne F M, Ru N, Isaacs W B. DD3: a new prostate-specific gene, highly overexpressed in prostate cancer. Cancer Res. 1999 Dec. 1; 59(23):5975-9). The mRNA expression analysis of said gene indicates a strong correlation between elevated levels of DD3 and the occurrence of cancer. The biological activity of said gene has to date not been ascertained, it has been speculated that it may be a non coding RNA. Nevertheless, analysis of the expression levels of said gene has the potential to be an excellent cancer marker.

However, mRNA analysis is not a technique that is suitable for application to a clinical and/or medium/high throughput laboratory setting. Its utility as a routine diagnostic tool is limited by the extreme instability of mRNA, and rapidly occurring expression changes following certain triggers (e.g. sample collection). Further, and most notably, large amount of mRNA are needed for the analysis (Lipshutz, R. J. et al., Nature Genetics 21:20-24, 1999; Bowtell, D. D. L. Nature genetics suppl. 21:25-32, 1999), which often cannot be obtained from a routine biopsy.

Furthermore, if it is confirmed that DD3 is indeed a non coding RNA, then protein and/or antibody assays will not be useful diagnostic tests. It follows that there exists a need for the farther investigation into the regulation of DD3 in order to identify a useful genomic marker that can be utilised in a clinical and/or laboratory setting. However, to date no single nucleotide polymorphisms or other genomic mutations are known of that may be used as markers for the development of DNA based assays.

It is anticipated that the development of such an assay would be of particular value in the improved detection and management of prostate cancer. Prostate cancer is a significant health care problem in Western countries with an incidence of 180 per 100 000 in the U.S. in 1999 (Cancer J Clin 1999; 49:8). Different screening strategies are employed to improve early detection, including determination of levels of prostate specific antigen (PSA) and digital rectal examination. If a prostate carcinoma is suspected in a patient, diagnosis of cancer is confirmed or excluded by the histological and cytological analysis of biopsy samples for features associated with malignant transformation. Particularly early stages of prostate carcinoma are often difficult to distinguish from benign hyperplasia of the prostate by routine histological examination even if an adequate biopsy is obtained (NcNeal J E et al., Hum Pathol 2001, 32:441-6). Furthermore, small or otherwise insufficient biopsy samples sometimes impede routine analysis.

Currently the most informative assay for the detection of prostate carcinomas is the analysis of prostate specific antigen levels. However, the utility of said assay is of limited value as the antigens are produced as the result of a general prostate immune response. In particular, when a modestly abnormal PSA value (4-10 ng/ml) is encountered in the context of a negative digital rectal exam (DRE), only 20-30% of individuals with such findings will demonstrate carcinoma on biopsy (Kantoff and Talcott, 8(3) Hematoll Oncol Clinics N Amer 555 (1994)). Within the last decade, numerous genes have been shown to be differentially expressed between benign hyperplastic prostate tumours and different grades of prostate cancer. However, no single marker has been shown to be sufficient for the distinction between the two lesions so far.

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulfite based cytosine methylation analysis. Nucleic Acids Res. 1996 Dec. 15; 24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April; 5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15; 25(12):2529-31, WO 95/00669) or by enzymatic digestion (Xiong Z, Laird P W. COBRA: a sensitive and quantitative DNA methylation assay. Nucleic Acids Res. 1997 Jun. 15; 25(12):2532-4). In addition, detection by hybridisation has also been described (Olek et al., WO 99/28498).

Further publications related to the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March; 6(3):387-95; Feil R, Charlton J, Bird A P, Walter J, Reik W. Methylation analysis on individual chromosomes: improved protocol for bisulfite genomic sequencing. Nucleic Acids Res. 1994 Feb. 25; 22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19; 157(1-2):261-4; WO 97/46705 and WO 95/15373.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. 1988 Oct. 15; 60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25; 23(8): 1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Aberrant DNA methylation within CpG islands is among the earliest and most common alterations in human malignancies leading to abrogation or overexpression of a broad spectrum of genes.

In addition, abnormal methylation has been shown to occur in CpG rich regulatory elements in intronic and coding parts of genes for certain tumours. In contrast to the specific hypermethylation of tumour suppresser genes, an overall hypomethylation of DNA can be observed in tumour cells. This decrease in global methylation can be detected early, far before the development of frank tumour formation. Also, correlation between hypomethylation and increased gene expression was reported for many oncogenes. In colon cancer, aberrant DNA methylation constitutes one of the most prominent alterations and inactivates many tumour suppresser genes such as p14ARF, p161NK4a, THBS1, MINT2, and MINT31 and DNA mis-match repair genes such as hMLH1.

### DESCRIPTION

The present invention provides methods for the analysis of the methylation status of the promoter and 5′ region of the gene DD3. Furthermore, the invention discloses genomic and chemically modified nucleic acid sequences derived from the gene DD3, as well as oligonucleotides and/or PNA-oligomers for analysis of cytosine methylation patterns within said gene. The present invention is based on the discovery that genetic and epigenetic parameters, in particular, the cytosine methylation patterns, of the gene DD3 are particularly suitable for the diagnosis and/or therapy of cell proliferative disorders.

The present invention provides methods for the analysis of cell proliferative disorders by means of analysis of the methylation of CpG dinucleotide positions that were heretofore not associated with the development of cancer. Furthermore, the invention discloses genomic and chemically modified nucleic acid sequences, as well as oligonucleotides and/or PNA-oligomers for analysis of cytosine methylation patterns within said region.

The objective of the invention can be achieved by analysis of the methylation state of the CpG dinucleotides within the genomic sequence according to Seq ID. No. 1 and sequences complementary thereto. Seq. ID No.1 discloses a fragment of the promoter and 5′ region of the gene DD3, wherein said region contains CpG dinucleotides exhibiting a disease specific methylation pattern. The methylation pattern of said fragment of the gene DD3 has heretofore not been analysed with regard to cell proliferative disorders. Due to the degeneracy of the genetic code, the sequence as identified in Seq. ID No. 1 should be interpreted so as to include all substantially similar and equivalent sequences upstream of the promoter region of a gene, which encodes a molecule with the biological activity and characteristics of DD3.

In a preferred embodiment of the method, the objective is achieved by analysis of a chemically modified nucleic acid containing a sequence of at least 18 bases in length according to one of Seq. ID No. 2 to Seq. ID No. 5 and sequences complementary thereto. Seq. ID No. 2 through 5 provide a modified version of the nucleic acid according to Seq. ID No. 1 wherein the modification of said sequence results in the synthesis of a sequence that is unique and distinct from Seq. ID No. 1. The nucleic acid molecules according to Seq. ID No. 1 to Seq. ID No. 5 could heretofore not be connected with the ascertainment of genetic and epigenetic parameters relevant to the analysis of cell proliferative disorders.

The object of the present invention can be further achieved by an oligonucleotide or oligomer for detecting the cytosine methylation state within pretreated DNA or genomic DNA according to Seq. ID No. 1 to Seq. ID No. 5. Said oligonucleotide or oligomer containing at least one base sequence having a length of at least 9 nucleotides which hybridises to a pretreated nucleic acid sequence according to Seq. ID No. 2 to Seq. ID No. 5 and sequences complementary thereto or to a genomic sequence comprising Seq. ID No. 1 and sequences complementary thereto. The oligonucleotides or oligomers according to the present invention constitute important and effective tools which, for the first time, make it possible to ascertain genetic and epigenetic parameters of the novel region as disclosed by the invention. The base sequence of said oligomers preferably contains at least one CG, TG or CA dinucleotide. The probes may also exist in the form of a PNA (peptide nucleic acid) which has particularly preferred pairing properties. Particularly preferred are oligonucleotides according to the present invention in which the cytosine of the CpG dinucleotide is within the middle third of the oligonucleotide, e.g. wherein the oligonucleotide is 13 bases in length the CG, TG or CA dinucleotide is positioned within the 5^{th}-9^{th} nucleotide from the 5′-end.

In a particularly preferred embodiment the sequence of said oligonucleotides is taken from the group comprising Seq. ID No. 6 to Seq. ID No. 92.

The oligomers according to the present invention are normally used in so called “sets” which contain at least one oligomer for the analysis of each of the CpG dinucleotides of a genomic sequence comprising Seq. ID No. 1 and sequences complementary thereto or to their corresponding CG, TG or CA dinucleotide within the pretreated nucleic acids according to Seq. ID No. 2 to Seq. ID No. 5 and sequences complementary thereto. Preferred is a set which contains at least one oligomer for each of the CpG dinucleotides within the gene DD3 in both the pretreated and genomic versions of said gene, Seq. ID No. 2 through 5 and Seq. ID No. 1 respectively. However, it is anticipated that for economic or other factors it may be preferable to analyse a limited selection of the CpG dinucleotides within said sequences and the contents of the set of oligonucleotides should be altered accordingly. Therefore, the present invention moreover relates to a set of at least 3 n (oligonucleotides and/or PNA-oligomers) used for detecting the cytosine methylation state in pretreated genomic DNA (Seq. ID No. 2 to Seq. ID No. 5 and sequences complementary thereto) and genomic DNA (Seq. ID No. 1 and sequences complementary thereto). These probes enable diagnosis and/or therapy of genetic and epigenetic parameters of cell proliferative disorders. The set of oligomers may also be used for detecting single nucleotide polymorphisms (SNPs) in pretreated genomic DNA (Seq. ID No. 2 to Seq. ID No. 5, and sequences complementary thereto) and genomic DNA (Seq. ID No. 1, and sequences complementary thereto).

Moreover, the present invention provides a set of at least two oligonucleotides which can be used as so-called “primer oligonucleotides” for amplifying DNA sequences of one of Seq. ID No. 1 to Seq. ID No. 5 and sequences complementary thereto, or segments thereof.

In the case of the sets of oligonucleotides according to the present invention, it is preferred that at least one and more preferably all members of the set of oligonucleotides are bound to a solid phase.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices may also be used.

Therefore, a further aspect of the present invention is a method for manufacturing an array fixed to a carrier material for analysis in connection with cell proliferative disorders, in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the analysis of cell proliferative disorders. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case corresponds to or are complementary to an 18 base long segment of the base sequences specified in the appendix (Seq. ID No. 1 to Seq. ID No. 5 and sequences complementary thereto), oligonucleotides and/or PNA-oligomers as well as instructions for carrying out and evaluating the described method. However, a kit along the lines of the present invention can also contain only part of the aforementioned components.

The present invention also provides methods for ascertaining genetic and/or epigenetic parameters of the gene DD3 within a subject by analysing cytosine methylation and single nucleotide polymorphisms. Said method comprising contacting a nucleic acid comprising one or more sequences from the group comprising Seq. ID No. 1 through Seq. ID No. 5 in a biological sample obtained from said subject with at least one reagent or a series of reagents, wherein said reagent or series of reagents, distinguishes between methylated and non methylated CpG dinucleotides within the target nucleic acid.

In a preferred embodiment said method comprises the following steps:

In the first step of the method a sample of the tissue to be analysed is obtained, this may be from any suitable sources such as cells or cell components, for example, cell lines, biopsies, blood, sputum, stool, urine, cerebrospinal fluid, tissue embedded in paraffin such as tissue from eyes, intestine, kidney, brain, heart, prostate, lung, colon, breast or liver, histologic object slides, or combinations thereof.

In the second step of the method genomic DNA is isolated from the sample. Extraction may be by means that are standard to one skilled in the art, these include the use of detergent lysates, sonification and vortexing with glass beads. Once the nucleic acids have been extracted the genomic double stranded DNA is used in the analysis.

In the third step of the method, the genomic DNA sample is treated in such a manner that cytosine bases which are umethylated at the 5′-position are converted to uracil, thymine, or another base which is dissimilar to cytosine in terms of hybridisation behaviour. This will be understood as ‘pretreatment’ hereinafter.

The above described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

In the fourth step of the method fragments of the pretreated DNA are amplified, using sets of primer oligonucleotides according to the present invention, and a preferably heat-stable polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR). The set of primer oligonucleotides includes at least two oligonucleotides whose sequences are each reverse complementary or identical to an at least 18 base-pair long segment of the base sequences specified in the appendix (Seq. ID No. 1 to Seq. ID No. 5 and sequences complementary thereto).

In a further embodiment of the method, the methylation status of preselected CpG positions within the nucleic acids comprising Seq ID No. 2 to Seq ID No. 5 can be detected by use of methylation specific primer oligonucleotides. This technique has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer which hybridises to a bisulfite treated CpG dinucleotide. Therefore the sequence of said primers comprises at least one CG, TG or CA dinucleotide. MSP primers specific for non methylated DNA contain a ‘T’ at the 3′ position of the C position in the CpG. According to the present invention, it is therefore preferred that the base sequence of said primers is required to comprise a sequence having a length of at least 9 nucleotides which hybridises to a pretreated nucleic acid sequence according to Seq. ID No. 2 to Seq. ID No. 5 and sequences complementary thereto wherein the base sequence of said oligomers comprises at least one CG, TG or CA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Wherein said labels are mass labels it is preferred that the labelled amplificates have a single positive or negative net charge for better detectability in the mass spectrometer. The detection may be carried out and visualised by means of matrix assisted laser desorption/ionization mass spectrometry (MALDI) or using electron spray mass spectrometry (ESI).

In the fifth step of the method the amplificates obtained during the fourth step of the method are analysed in order to ascertain the methylation status of the CpG dinucleotides prior to the treatment.

Wherein the amplificates were obtained by means of MSP amplification the presence or absence of an amplificate is in itself indicative of the methylation state of the CpG positions covered by the primer, according to the base sequences of said primer.

Amplificates obtained by means of both standard and methylation specific PCR may be further analysed by means of hybridisation based methods such as, but not limited to, array technology and probe based technologies as well as by means of techniques such as sequencing and template directed extension.

In one embodiment of the method, the amplificates synthesised in step 4 are subsequently hybridised to an array or a set of oligonucleotides and/or PNA probes. In this context, the hybridisation takes place in the manner described in the following. The set of probes used during the hybridisation is preferably composed of at least 2 oligonucleotides or PNA-oligomers. In the process, the amplificates serve as probes which hybridise to oligonucleotides previously bonded to a solid phase. The non-hybridised fragments are subsequently removed. Said oligonucleotides contain at least one base sequence having a length of at least 9 nucleotides which is reverse complementary or identical to a segment of the base sequences specified in the appendix, the segment comprises at least one CG, TG or CA dinucleotide. In a preferred embodiment said dinucleotide is present in the central third of the oligomer. For example, wherein the oligomer comprises one CG dinucleotide, said dinucleotide is preferably the 5^{th} to 9^{th} nucleotide from the 5′-end of a 13-mer. One oligonucleotide exists for the analysis of each CpG dinucleotide within the sequence according to Seq. ID No. 1, and the equivalent positions within Seq. ID No. 2 to Seq. ID No. 5. Said oligonucleotides may also be present in the form of peptide nucleic acids. The non-hybridised amplificates are then removed.

In the final step of the method, the hybridised amplificates are detected. In this context, it is preferred that labels attached to the amplificates are identifiable at each position of the solid phase at which an oligonucleotide sequence is located.

In a further embodiment of the method, the methylation status of the CpG positions (prior to treatment) may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which is designed to hybridise to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment it is required that the probe be methylation specific, as described in U.S. Pat. No. 6,331,393, (hereby incorporated by reference) also known as the Methyl Light assay. Variations on the TaqMan™ detection methodology that are also suitable for use with the described invention include the use of dual probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids is the use of blocker oligonucleotides (also known as the ‘Heavy Methyl’ assay). The use of such oligonucleotides has been described in BioTechniques 23(4), 1997, 714-720 D. Yu, M. Mukai, Q. Liu, C. Steinman. Blocking probe oligonucleotides are hybridised to the bisulfite treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridise to the bisulfite treated nucleic acid in a methylation status specific manner. For example, for detection of methylated nucleic acids within a population of unmethylated nucleic acids suppression of the amplification of nucleic acids which are unmethylated at the position in question would be carried out by the use of blocking probes comprising a ‘CG’ at the position in question, as opposed to a ‘CA’.

In a further preferred embodiment of the method the fifth step of the method is carried out by the use of template directed oligonucleotide extension, such as MsSNuPE as described by Gonzalgo and Jones (Nucleic Acids Res. 25:2529-2531).

In a further embodiment of the method the fifth step of the method is enabled by sequencing and subsequent sequence analysis of the amplificate generated in the third step of the method (Sanger F., et al., 1977 PNAS USA 74: 5463-5467).

A further embodiment of the method according to the invention is a method for the analysis of the methylation status of genomic DNA according to the invention (SEQ ID No. 1) without the need for pretreatment.

In the first step of the method the genomic DNA sample must be isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis. In a preferred embodiment the DNA may be cleaved prior to the treatment, this may be any means standard in the state of the art, in particular with restriction endonucleases.

In the second step, the DNA is then digested with one or more methylation sensitive restriction enzymes. The digestion is carried out such that hydrolysis of the DNA at the restriction site is informative of the methylation status of a specific CpG dinucleotide.

In the third step, which is optional but a preferred embodiment, the restriction fragments are amplified. This is preferably carried out using a polymerase chain reaction.

In the final step, the amplificates are detected. The detection may be by any means standard in the art, for example, but not limited to, gel electrophoresis analysis, hybridisation analysis, incorporation of detectable tags within the PCR products, DNA array analysis, MALDI or ESI analysis. Suitable labels for use in the detection of the digested nucleic acid fragments include fluorophore labels, radionuclides and mass labels as described above.

The oligomers according to the present invention or arrays thereof as well as a kit according to the present invention are intended to be used for the diagnosis and/or therapy of cell proliferative disorders. According to the present invention, the method is preferably used for the diagnosis and/or therapy of cell proliferative disorders by analysis of important genetic and/or epigenetic parameters within the disclosed 5′ and promoter region of the gene DD3.

The methods according to the present invention are used, for example, for the diagnosis and/or therapy of cell proliferative disorders.

The nucleic acids according to the present invention Seq. ID No. 1 to Seq. ID No. 5 and sequences complementary thereto can be used for the diagnosis and/or therapy of genetic and/or epigenetic parameters associated with the gene DD3.

The present invention moreover relates to a method for manufacturing a diagnostic agent and/or therapeutic agent for the diagnosis and/or therapy of diseases associated with the gene DD3 by analysing methylation patterns of said gene, the diagnostic agent and/or therapeutic agent being characterised in that at least one nucleic acid according to the present invention is used for manufacturing it, possibly together with suitable additives and auxiliary agents.

A further subject matter of the present invention relates to a diagnostic agent and/or therapeutic agent for diseases associated with the gene DD3 by analysing methylation patterns of said gene, the diagnostic agent and/or therapeutic agent containing at least one nucleic acid according to the present invention, possibly together with suitable additives and auxiliary agents.

An accordingly preferred embodiment of the described methods for the analysis of methylation within the genomic and treated nucleic acids disclosed in Seq ID NO. 1 to Seq. ID No. 5 is the application of said methods in a clinical setting. One aspect of the present invention is the use of the disclosed methods and nucleic acids for the improved detection and management of prostate tumours. Therefore, in a preferred embodiment of the method analysis of patient methylation patterns is carried out in combination with the analysis of prostate serum antigen levels. PSA diagnostic kits are commercially available; for example PROS-CHECK PSA, from Yang Laboratories, Inc. Bellevue, Wash.; Hybritech Tandem-E and Hybritech Tandem-R, from Hybritech, Inc., La Jolla, Calif.; Abbott Imx PSA Assay, from Abbott Laboratories, Abbott Park, Ill.; and ACS PSA Assay, from Ciba-Corning Diagnostics Corporation, East Walpole, Mass.

The present invention moreover relates to the diagnosis and/or prognosis of events which are disadvantageous to patients or individuals in which important genetic and/or epigenetic parameters within the gene DD3 may be used as markers. Said parameters obtained by means of the present invention may be compared to another set of genetic and/or epigenetic parameters, the differences serving as the basis for a diagnosis and/or prognosis of events which are disadvantageous to patients or individuals.

Moreover, a subject matter of the present invention is a kit which may be composed, for example, of a bisulfite-containing reagent, a set of primer oligonucleotides containing at least two oligonucleotides whose sequences in each case correspond or are complementary to a 18 base long segment of the base sequences specified in the appendix (Seq ID NO 1 to Seq ID NO 5), oligonucleotides and/or PNA-oligomers as well as instructions for carrying out and evaluating the described method. In a further preferred embodiment said kit may further comprise standard reagents for performing a CpG position specific methylation analysis wherein said analysis comprises one or more of the following techniques: MS-SNuPE, MSP, Methyl light, Heavy Methyl, and nucleic acid sequencing. However, a kit along the lines of the present invention can also contain only part of the aforementioned components.

In the context of the present invention the term “hybridisation” is to be understood as a bond of an oligonucleotide to a completely complementary sequence along the lines of the Watson-Crick base pairings in the sample DNA, forming a duplex structure. To be understood by “stringent hybridisation conditions” are those conditions in which a hybridisation is carried out at 60° C. in 2.5×SSC buffer, followed by several washing steps at 37° C. in a low buffer concentration, and remains stable.

In the context of the present invention, “genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations. Further epigenetic parameters include, for example, the acetylation of histones which, however, cannot be directly analysed using the described method but which, in turn, correlates with the DNA methylation.

The use of multiple CpG sites from a diverse “panel” of such sites that are present in the gene DD3 in addition allows for a relatively high degree of sensitivity and specificity in comparison to a single CpG site-diagnostic and/or detection tool. Furthermore, the panel as described herein may be adapted for use in the analysis of multiple diseases all affected by DD3, such as cancer, in particular prostate cancer.

Although the present invention has been fully described herein, it is to be noted that various changes and modifications are apparent to those skilled in the art. Such changes and modifications are to be understood as included within the scope of the present invention as defined by the appended claims. The invention will now be further described with respect to the following examples without being limited to these.

### EXAMPLE 1

### Methylation Analysis of the DD3 Gene Promoter Region

The following example relates to a fragment of the promoter region of the gene DD3 in which the methylation status of a specific CG-position is bisulfite treated and analysed using two different methods.

DNA Isolation and Bisulfite Treatment.

Briefly, genomic DNA was isolated from human prostate cells by standard methods using the Qiagen extraction kit. The DNA from each sample was treated using a bisulfite solution (hydrogen sulfite, disulfite) according to the agarose bead method (Olek et al 1996). The treatment is such that all non methylated cytosines within the sample are converted to thymine, conversely 5-methylated cytosines within the sample remain unmodified. After bisulfite treatment, the DNA was then analysed either by means of MS SNuPE as described by Gonzalgo and Jones (Nucleic Acids Res. 25:2529-2531) or by means of fluorescent oligonucleotide hybridisation analysis.

MsSNuPE Reactions.

PCR amplification of the bisulfite converted DNA was performed using primers specific for the CpG islands of interest, and detection was performed using additional specific primers (extension probes).

In the first step, a 560 bp long fragment of the DD3 gene is amplified in a polymerase chain reaction by means of primers: tggttaattttattgaatgg (Seq. ID No.93) and aaacaaatacaccaccaactta (Seq. ID No.94).

The amplificate is then analysed by means of MsSNuPE extension probes located immediately 5′ of the CpG to be analysed, the sequences being: attggtgttatagagtta (Seq. ID No.95).

A pair of reactions were set up for each sample using either 2′,3′-didesoxycytidine triphosphate (ddCTP) or 2′,3′-didesoxythymidine triphosphate (ddTTP) for single nucleotide extension. The terminating triphosphates may be labelled, for example, with two different dyes. This makes the elongation products distinguishable from each other. These different labels may, for example, be absorbing dyes such as Megaprime™ for ddTTP or Rediprime II™ for ddCTP.

The MsSNuPE extension probes are hybridised to the amplificate. Extension of the primers was then carried out by means of a polymerase enzyme. If a methylated cytosine was present, the elongation product attggtgttatagagttac is produced whereas the elongation product attggtgttatagagttat is produced if a non-methylated cytosine is present at the site to be analysed. Thus, different elongation products arise depending on the methylation status of the specific cytosine.

The extended MsSNuPE primers (probes) were then analysed by means of polyacrylamide gel electrophoresis.

Hybridisation Analysis

Subsequent to the bisulfite treatment, the treated DNA sample is diluted with water or an aqueous solution. Preferably, the DNA is subsequently desulfonated. The DNA sample is then amplified in a polymerase chain reaction, preferably using a heat-resistant DNA polymerase. In the present case, cytosines of the gene DD3 are analysed. To this end, a defined fragment having a length of 560 bp is amplified with the specific primer oligonucleotides tggttttaattttattgaatgg (Seq. ID No. 93) and aaacaaatacaccaccaactta (Seq. ID No. 94).

The amplificate serves as a sample which hybridises to an oligonucleotide previously bound to a solid phase, forming a duplex structure, for example tgtgttaaacgatgtgaa (Seq. ID No. 32, the cytosine to be detected being located at position 139 of the amplificate. The detection of the hybridisation product is based on Cy3 and Cy5 fluorescently labelled primer oligonucleotides which have been used for the amplification. A hybridisation reaction of the amplified DNA with the oligonucleotide takes place only if a methylated cytosine was present at this location in the bisulfite-treated DNA. Thus, the methylation status of the specific cytosine to be analysed is inferred from the hybridisation product.

In order to verify the methylation status of the position, a sample of the amplificate is further hybridised to another oligonucleotide previously bonded to a solid phase. Said olignonucleotide is identical to the oligonucleotide previously used to analyse the methylation status of the sample, with the exception of the position in question. At the position to be analysed said oligonucleotide comprises a thymine base as opposed to a cytosine base i.e tgtgttaaatgatgtgaa (Seq. ID No. 33). Therefore, the hybridisation reaction only takes place if an unmethylated cytosine was present at the position to be analysed.

### EXAMPLE 2

### Identification of the Methylation Status of a CpG Site within Seq ID No. 1

A fragment of the upstream region of the gene DD3 (Seq ID No. 1) was PCR amplified using primers aagtgagccataacaagcat (Seq. ID No. 96) and CTTTTGTGTCATCCCAGTTC (Seq. ID No. 97). The resultant amplificate (170 bp in length) contained an informative CpG at position 62. The amplificate DNA was digested with the restriction endonuclease HgaI, recogniton site GACGC. Hydrolysis by said endonuclease is blocked by methylation of the CpG at position 62 of the amplificate. The digest was used as a control.

Genomic DNA was isolated from samples using the DNA wizard DNA isolation kit (Promega). Each sample was digested using HgaI according to manufacturer's recommendations (New England Biolabs).

10 ng of each genomic digest was then amplified using PCR primers aagtgagccataacaagcat (Seq. ID No. 96) and cttttgtgtcatcccagttc (Seq. ID No. 97). The PCR reactions were performed using a thermocycler (Eppendorf GmbH) using 10 ng of DNA, 6 pmole of each primer, 200 μM of each dNTP, 1.5 mM MgC12 and 1 U of HotstartTaq (Qiagen AG). The other conditions were as recommended by the Taq polymerase manufacturer. Using the above mentioned primers, gene fragments were amplified by PCR performing a first denaturation step for 14 min at 96° C., followed by 30-45 cycles (step 2: 60 sec at 96° C., step 3: 45 sec at 52° C., step 4: 75 sec at 72° C.) and a subsequent final elongation of 10 min at 72° C. The presence of PCR products was analysed by agarose gel electrophoresis. Wherein the position in question was methylated, PCR products were detectable after 30 cycles.

1. A method for analysis diagnosis or prognosis of a cell proliferative disorder comprising: contacting genomic DNA obtained from a test subject with at least one reagent or a series of reagents that distinguishes between methylated and non methylated CpG dinucleotides; and determining the methylation state of as least one CpG dinucleotide of SEQ ID NO: 1, whereby a diagnosis or prognosis of a cell proliferative disorder is, at least in part, afforded.

2. The method of claim 1, wherein determining the methylation state comprises analysis of at lease one sequence, or contiguous portion thereof, selected from the group consisting of SEQ ID NOS:2-5.

3. The method of claim 1, wherein the genomic DNA is obtained or derived from prostate cells.

4. A nucleic acid molecules comprising at least 18 contiguous bases of a sequence selected from the group consisting of SEQ ID NOS:1-5, and sequences complementary thereto.

5. An oligomer or peptide nucleic acid (PNA)-oligomer, said oligomer in each case consisting essentially of at least one base sequence at least 10 contiguous bases of a sequence selected from the group consisting of SEQ ID NOS:1-5, and sequences complimentary thereto.

6. The oligomer of claim 5, consisting essentially of a sequence selected from the group consisting of SEQ ID NOS:6-92.

7. The oligomer of in claim 5, comprising at least one CpG dinucleotide.

8. The oligomer of in claim 7, characterised in that the cytosine of the CpG dinucleotide is located approximately in the middle third of the oligomer.

9. A set of oligomers, comprising at least two oligomers according to any one of claims 5 to 8.

10. The set of oligomers of claim 9, comprising oligomers suitable for detecting the methylation state of all CpG dinucleotides within SEQ ID NO: 1, and sequences complementary thereto.

11. The set of oligomers of claim 9, comprising oligomers suitable as primer oligonucleotides for amplification of a sequence, or portion thereof, of a sequence selected from SEQ ID NOS: 1-5, and sequences complementary thereto.

12. The set of oligonucleotides of claim 9, wherein at least one oligonucleotide is bound to a solid phase.

13. A method for determining methylation state of single nucleotide polymorphisms, comprising using a set of oligonucleotides comprising at least three oligomers according to any one of claims 5 through 12 in at least one assay suitable for detecting at least one of a cytosine methylation state, and a single nucleotide polymorphisms (SNPs) within a sequence selected from the group consisting of SEQ ID NOS:1-5, and sequences complementary thereto.

14. (canceled)

15. An arrangement of different oligomers according to any one of claims 5 through 12, coupled to a solid phase.

16. The array of claim 15, wherein the oligomers are arranged in the form of a rectangular or hexagonal lattice.

17. The array of claim 15, wherein the solid phase comprises silicon, glass, polystyrene, aluminium, steel, iron, copper, nickel, silver, gold, and combinations thereof.

18. (canceled)

19. A method for analysis diagnosis or prognosis of a cell proliferative disorder comprising:
a) obtaining, from a test subject, a biological sample containing genomic DNA,
b) isolating the genomic DNA,
c) converting, by chemically treating the genomic DNA or a portion thereof, cytosine bases that are unmethylated at the 5-position to uracil or another base which is dissimilar to cytosine in terms of hybridisation behaviour;
d) amplifying the chemically pretreated genomic DNA, or a portion thereof, using a sets of primer oligonucleotides according to any one of claims 11 or 12 and a polymerase; and
e) determining, based at least in part on the amplifying, the methylation status of one or more cytosine positions within a sequence selected from the group consisting of SEQ ID NOS:1-5, and sequences complementary thereto, whereby analysis, diagnosis or prognosis of a cell proliferative disorder is, at least in part, afforded.

20. The method of claim 19, wherein determining in e) is by means of hybridisation of at least one oligonucleotide according to any one of claims 5 to 12.

21. The method of claim 19, wherein determining in e) is by means of hybridisation of at least one oligonucleotide according to any one of claims 5 to 12, and extension of said hybridised oligonucleotide(s) by at least one nucleotide base.

22. The method of claim 19, wherein determining in e) is by means of sequencing.

23. The method of claim 19, wherein amplifying in d) comprises use of methylation specific primers.

24. The method of claim 19, comprising a combination of at least two of the methods described in any one of claims 20 to 23.

25. The method of claim 19, wherein converting comprises use of an agent selected from the group consisting of as recited in claim 19, characterised in that the chemical treatment is carried out by means of a solution of a bisulfite, hydrogen sulfites, disulfite, and combinations thereof.

26. A method for analysis, diagnosis or prognosis of a cell proliferative disorder, comprising:
a) obtaining, from a test subject, a biological sample containing genomic DNA,
b) isolating the genomic DNA,
c) digesting the isolated genomic DNA, or a portion thereof, comprising SEQ ID NO:1 with one or more methylation-sensitive restriction enzymes; and
d) determining, by detecting one or more of the DNA fragments generated in c), the methylation state of at least one CpG dicleotide of SEQ ID NO: 1, whereby analysis, diagnosis or prognosis of a cell proliferative disorder is, at least in part, afforded.

27. The method of claim 26, further comprising, prior to d), amplification of the DNA digest.

28. The method of any one of claims 19 or 27, wherein more than ten different fragments having a length of about 100 to about 2000 nucleotides are amplified.

29. The method of claim 28, wherein the amplification is carried out in one reaction vessel.

30. The method of any one of claims 19 or 27, wherein the polymerase is a heat-resistant DNA polymerase.

31. The method of any one of claims 19 or 27, wherein the amplification is carried out by polymerase chain reaction (PCR) means.

32. The method of any one of claims 19 or 27, wherein the amplificates carry detectable labels.

33. The method of claim 32, wherein the labels are selected from the group consisting of fluorescence labels, radionuclides, detachable molecule fragments having a typical mass which can be detected in a mass spectrometer, and combinations thereof.

34. The method of claim 33, wherein amplificates or fragments of the amplificates are detected in the mass spectrometer.

35. The method of claim 34, wherein the produced fragments have a single positive or negative net charge for better detectability in the mass spectrometer.

36. The method of claim 34, wherein detection is carried out and visualised by means of at least one of matrix assisted laser desorption/ionisation mass spectrometry (MALDI), and electron spray mass spectrometry (ESI).

37. The method of any one of claims 19 and 27, wherein the genomic DNA is obtained from a source selected from the group consisting of cells or cellular components which contain DNA, cell lines, histological slides, biopsies, tissue embedded in paraffin, and combinations thereof.

38. A kit comprising a reagent having at least one of bisulfite, disufite, and hydrogen sulfite, as well as oligomers or PNA-oligomers according to any one of the claims 5 to 13.

39. The kit of claim 38, further comprising standard reagents for performing a methylation assay from the group consisting of MS-SNuPE, MSP, MethyLight, Heavy Methyl, nucleic acid sequencing, and combinations thereof.

40. (canceled)

41. (canceled)


SEQUENCE LISTING (97 sequences)

SEQ ID NO: 1 - DNA, 3581 bp, Homo Sapiens
acaaaagacttagtcaatttaatttgtaagaattctacttttgtagacctgaactggatacaattttatctctggacatcaaagcccggggtcaaatgattgcttttctcttttgctgggagtttttgttgtcatgttttatactttagacattttacttttggtcactaggtggctctgaatttccattagaaagtgaaagtctttagaaaacactggaaaacatacataattttcaccaaataattttaaaatacttcattcaacaatgattgacttgagaatattaatcaaagtttatcaaaagacccataaactccaaatagcactctgtcaatctatcttatgtagattttgagatagattatttatagctttacatgaattatagcttttgcattggttataggtacaatttttaaatgattcataaactctgttccaaataatgccactctggagctatatcatattatgtatcatgtatggtcaaacaactgggtccgtcatatttaaaagacaacttcaaaatactgtcatataactttaatggctttaaagtctttcctaagtgtactttagcagcctccaatgcatctggtgccaactctgaaatcatttttaaggttagcaattttaaacaaaaagaatggttagaacaaggacctttagccagtttggaagtcaccaacagtttccttctgttgttggaaaaagtgatttctttgccctgagttctacaacaaccattaaatgtgctgcatttgactcctgtaaagatttaacttttttccaaatctttaatgacaatactctctgttatttcatcaaaaagtgtgttaacataggaagcagtgatccaaagtcatttccggataacctattaataagggaggcaggaatggcggttgcagaaatgtgcgcagagagagcaactaccatgggaataatccagatggcggtctgcttggctcgaactttctggaagaggcccatctttaaaatgcttaataccactttttactatagattccacacatgttcatttcttaacaaactctgagagaggtggtgagatactcttgcagtaattttacccccagccccctggaccatccttttcaaactctccaaactctccacgtttctttttacatgttcaaaatgcaggcagtgtagaatgactgtctgtggctcctccagtcctagctcttgtatggaggcggaggtccctacatacacgaccacatgagattgagctccagagtcaggcccttcgccatgagcatggctgtggacacgtaaggagcctcagtggctccacaacttggcaatcttaatccaaacaaaccaggtaaaacgtttttagttggactcaagataaatcccaggcagagcaatcctcaaaagaaaccagcaaactggatatttggtttctgatttacaatttttttaaaacagacaaaatagcttccttcactccccactgtgtctttggctccaacctcattgaatggttgccatttctgagacacatcctctaccgcaaacctgcactcctcccacctcgtgcaccatctccccttctagctccatctggcaatgtgtacttttcctcaagtcctgtgtcaaacgatgtgaagactgaggtcttcttctggtcggggcagcattatagcatcatgacaaaaagcccaggtgctgcctgcactggaagtcaagtcaattactggtcatgagatcttgagcaagttacttaatttcagttgccacatccataaaatgggatcatagcaggactactggtgtcacagagttacgtgaggtttaaataagttaccttaaaggacttagtacagtacctggtatactatgtggcttaagtattaataggctttgcagtaagtgtagtcatctaagtgagccataacaagcataggcgtgcttctgggagtgctcaccccaagtctgaattagacgcaactactctctactctcctggagaaaacatactcgtaataccacttcaaacacaagctggtggtgtacttgcccagttctaccaatgaactgggatgacacaaaagtaatgaacagatgaagtttcatatcccccgtccccaagagaggatctggtgtcgtctatttagcttccttgcctctcctgtgctctctgaggactgagagattaaaagccacgttggtggatactgcagaagagcaggtggggaggagaatggtatggcagtgacaggaagtgctgggaggcccaatatggagaaggagaggggtagtggggaagagggagtgggataaagggcatggggaagcatggggaagagggagctttcttctgtgtttagacagtgagaggtgcccatgagtcaataaataaaagaggaaaggaaatttaatgagttgccatggactaagcacttcgattgagttacactgtttgaaagatattagtaaagatgggaactcacatttggacaagacttcactagaggagcaccttaggaattgacctgtggatctcaacttcgttagggttaaaagattatttgttgggcaagggtaggaccaataacctcattcacaatgcattcattgattcgttgattcacagagcaaatacttctgaacaactcctgtgtttctggcactgttctaggcaccagtgatataggagccaacaagacagacatgtcactgctctcatggagctgcatttcagtgcatggaggcagaaaacaaacaaacaaataaataaataaataaataaataagataatttttaatagcaacgtgtcaacatagtgtgacgggaaggagcatgatgagacagaaggaaggtttaaactgggaaatctgagaaatggtatggttgtatgtgggttggcattcttgcatgatgggagtggccacctgctttcatattctgaagtcagagtgttccagacagaagaaatagcaagtgccgagaagctggcatcagaaaaacagaggggagatttgtgtggctgcagccgagggagaccaggaagatctgcatggtgggaaggacctgatgatacagaggtctgtaggccatgggaatgggtttggaattttattcaaagagctatgggaagtgactagaaggtttaaagttggggaagaggttttgtgtttctgttatatttgtgttttatacaaattactctggttgctgtgttgataggacagcagaagggtaggagcagggacaccagttaagttattgcaatggttaaggtgagaggtggtggctgggcctaggcctttttggagtgaacaagctatttgcttggcttccatgatccttctccctccactggaatggaaggtacttgatattagagatttttgtctgtttcatcactactgtaaccccaatgtctacaacagactctggcacatagaagacacttgtttaatgttgctgactaactatactgagagagaagaatgatgcagagcagatttagagaaaaagtcaaaggttctgtttagcacatgttaaatttgagatacccattagatatttaaatggagatatc

SEQ ID NO: 2 - DNA, 3581 bp, Artificial Sequence
  chemically treated genomic DNA (Homo sapiens)
ataaaagatttagttaatttaatttgtaagaattttatttttgtagatttgaattggatataattttatttttggatattaaagttcggggttaaatgattgttttttttttttgttgggagtttttgttgttatgttttatattttagatattttatttttggttattaggtggttttgaatttttattagaaagtgaaagtttttagaaaatattggaaaatatatataatttttattaaataattttaaaatattttatttaataatgattgatttgagaatattaattaaagtttattaaaagatttataaattttaaatagtattttgttaatttattttatgtagattttgagatagattatttatagttttatatgaattatagtttttgtattggttataggtataatttttaaatgatttataaattttgttttaaataatgttattttggagttatattatattatgtattatgtatggttaaataattgggttcgttatatttaaaagataattttaaaatattgttatataattttaatggttttaaagttttttttaagtgtattttagtagtttttaatgtatttggtgttaattttgaaattatttttaaggttagtaattttaaataaaaagaatggttagaataaggatttttagttagtttggaagttattaatagtttttttttgttgttggaaaaagtgatttttttgttttgagttttataataattattaaatgtgttgtatttgatttttgtaaagatttaatttttttttaaatttttaatgataatattttttgttattttattaaaaagtgtgttaatataggaagtagtgatttaaagttattttcggataatttattaataagggaggtaggaatggcggttgtagaaatgtgcgtagagagagtaattattatgggaataatttagatggcggtttgtttggttcgaattttttggaagaggtttatttttaaaatgtttaatattattttttattatagattttatatatgtttattttttaataaattttgagagaggtggtgagatatttttgtagtaattttatttttagttttttggattattttttttaaattttttaaattttttacgtttttttttatatgtttaaaatgtaggtagtgtagaatgattgtttgtggtttttttagttttagtttttgtatggaggcggaggtttttatatatacgattatatgagattgagttttagagttaggtttttcgttatgagtatggttgtggatacgtaaggagttttagtggttttataatttggtaattttaatttaaataaattaggtaaaacgtttttagttggatttaagataaattttaggtagagtaatttttaaaagaaattagtaaattggatatttggtttttgatttataatttttttaaaatagataaaatagttttttttattttttattgtgtttttggttttaattttattgaatggttgttatttttgagatatattttttatcgtaaatttgtattttttttatttcgtgtattatttttttttttagttttatttggtaatgtgtatttttttttaagttttgtgttaaacgatgtgaagattgaggtttttttttggtcggggtagtattatagtattatgataaaaagtttaggtgttgtttgtattggaagttaagttaattattggttatgagattttgagtaagttatttaattttagttgttatatttataaaatgggattatagtaggattattggtgttatagagttacgtgaggtttaaataagttattttaaaggatttagtatagtatttggtatattatgtggtttaagtattaataggttttgtagtaagtgtagttatttaagtgagttataataagtataggcgtgtttttgggagtgtttattttaagtttgaattagacgtaattattttttatttttttggagaaaatatattcgtaatattattttaaatataagttggtggtgtatttgtttagttttattaatgaattgggatgatataaaagtaatgaatagatgaagttttatatttttcgtttttaagagaggatttggtgtcgtttatttagtttttttgttttttttgtgttttttgaggattgagagattaaaagttacgttggtggatattgtagaagagtaggtggggaggagaatggtatggtagtgataggaagtgttgggaggtttaatatggagaaggagaggggtagtggggaagagggagtgggataaagggtatggggaagtatggggaagagggagtttttttttgtgtttagatagtgagaggtgtttatgagttaataaataaaagaggaaaggaaatttaatgagttgttatggattaagtatttcgattgagttatattgtttgaaagatattagtaaagatgggaatttatatttggataagattttattagaggagtattttaggaattgatttgtggattttaatttcgttagggttaaaagattatttgttgggtaagggtaggattaataattttatttataatgtatttattgattcgttgatttatagagtaaatatttttgaataatttttgtgtttttggtattgttttaggtattagtgatataggagttaataagatagatatgttattgtttttatggagttgtattttagtgtatggaggtagaaaataaataaataaataaataaataaataaataaataagataatttttaatagtaacgtgttaatatagtgtgacgggaaggagtatgatgagatagaaggaaggtttaaattgggaaatttgagaaatggtatggttgtatgtgggttggtatttttgtatgatgggagtggttatttgtttttatattttgaagttagagtgttttagatagaagaaatagtaagtgtcgagaagttggtattagaaaaatagaggggagatttgtgtggttgtagtcgagggagattaggaagatttgtatggtgggaaggatttgatgatatagaggtttgtaggttatgggaatgggtttggaattttatttaaagagttatgggaagtgattagaaggtttaaagttggggaagaggttttgtgtttttgttatatttgtgttttatataaattattttggttgttgtgttgataggatagtagaagggtaggagtagggatattagttaagttattgtaatggttaaggtgagaggtggtggttgggtttaggtttttttggagtgaataagttatttgtttggtttttatgatttttttttttttattggaatggaaggtatttgatattagagatttttgtttgttttattattattgtaattttaatgtttataatagattttggtatatagaagatatttgtttaatgttgttgattaattatattgagagagaagaatgatgtagagtagatttagagaaaaagttaaaggttttgtttagtatatgttaaatttgagatatttattagatatttaaatggagatatt

SEQ ID NO: 3 - DNA, 3581 bp, Artificial Sequence
  chemically treated genomic DNA (Homo sapiens)
gatatttttatttaaatatttaatgggtattttaaatttaatatgtgttaaatagaatttttgattttttttttaaatttgttttgtattattttttttttttagtatagttagttagtaatattaaataagtgttttttatgtgttagagtttgttgtagatattggggttatagtagtgatgaaatagataaaaatttttaatattaagtattttttattttagtggagggagaaggattatggaagttaagtaaatagtttgtttattttaaaaaggtttaggtttagttattattttttattttaattattgtaataatttaattggtgtttttgtttttatttttttgttgttttattaatatagtaattagagtaatttgtataaaatataaatataatagaaatataaaattttttttttaattttaaattttttagttattttttatagttttttgaataaaattttaaatttatttttatggtttatagatttttgtattattaggttttttttattatgtagatttttttggtttttttcggttgtagttatataaattttttttttgtttttttgatgttagtttttcggtatttgttattttttttgtttggaatattttgattttagaatatgaaagtaggtggttatttttattatgtaagaatgttaatttatatataattatattattttttagattttttagtttaaattttttttttgttttattatgttttttttcgttatattatgttgatacgttgttattaaaaattattttatttatttatttatttatttatttgtttgtttgttttttgtttttatgtattgaaatgtagttttatgagagtagtgatatgtttgttttgttggtttttatattattggtgtttagaatagtgttagaaatataggagttgtttagaagtatttgttttgtgaattaacgaattaatgaatgtattgtgaatgaggttattggttttatttttgtttaataaataattttttaattttaacgaagttgagatttataggttaatttttaaggtgtttttttagtgaagttttgtttaaatgtgagtttttatttttattaatattttttaaatagtgtaatttaatcgaagtgtttagtttatggtaatttattaaattttttttttttttttatttattgatttatgggtattttttattgtttaaatatagaagaaagtttttttttttttatgtttttttatgttttttattttatttttttttttttattatttttttttttttttatattgggttttttagtattttttgttattgttatattatttttttttttatttgtttttttgtagtatttattaacgtggtttttaattttttagtttttagagagtataggagaggtaaggaagttaaatagacgatattagatttttttttggggacgggggatatgaaattttatttgtttattatttttgtgttattttagtttattggtagaattgggtaagtatattattagtttgtgtttgaagtggtattacgagtatgttttttttaggagagtagagagtagttgcgtttaatttagatttggggtgagtatttttagaagtacgtttatgtttgttatggtttatttagatgattatatttattgtaaagtttattaatatttaagttatatagtatattaggtattgtattaagttttttaaggtaatttatttaaattttacgtaattttgtgatattagtagttttgttatgattttattttatggatgtggtaattgaaattaagtaatttgtttaagattttatgattagtaattgatttgatttttagtgtaggtagtatttgggttttttgttatgatgttataatgttgtttcgattagaagaagattttagtttttatatcgtttgatataggatttgaggaaaagtatatattgttagatggagttagaaggggagatggtgtacgaggtgggaggagtgtaggtttgcggtagaggatgtgttttagaaatggtaattatttaatgaggttggagttaaagatatagtggggagtgaaggaagttattttgtttgttttaaaaaaattgtaaattagaaattaaatatttagtttgttggttttttttgaggattgttttgtttgggatttattttgagtttaattaaaaacgttttatttggtttgtttggattaagattgttaagttgtggagttattgaggttttttacgtgtttatagttatgtttatggcgaagggtttgattttggagtttaattttatgtggtcgtgtatgtagggattttcgtttttatataagagttaggattggaggagttatagatagttattttatattgtttgtattttgaatatgtaaaaagaaacgtggagagtttggagagtttgaaaaggatggtttagggggttgggggtaaaattattgtaagagtattttattattttttttagagtttgttaagaaatgaatatgtgtggaatttatagtaaaaagtggtattaagtattttaaagatgggttttttttagaaagttcgagttaagtagatcgttatttggattatttttatggtagttgttttttttgcgtatatttttgtaatcgttatttttgttttttttattaataggttattcggaaatgattttggattattgttttttatgttaatatattttttgatgaaataatagagagtattgttattaaagatttggaaaaaagttaaatttttataggagttaaatgtagtatatttaatggttgttgtagaatttagggtaaagaaattattttttttaataatagaaggaaattgttggtgatttttaaattggttaaaggtttttgttttaattattttttttgtttaaaattgttaattttaaaaatgattttagagttggtattagatgtattggaggttgttaaagtatatttaggaaagattttaaagttattaaagttatatgatagtattttgaagttgttttttaaatatgacggatttagttgtttgattatatatgatatataatatgatatagttttagagtggtattatttggaatagagtttatgaattatttaaaaattgtatttataattaatgtaaaagttataatttatgtaaagttataaataatttattttaaaatttatataagatagattgatagagtgttatttggagtttatgggttttttgataaattttgattaatatttttaagttaattattgttgaatgaagtattttaaaattatttggtgaaaattatgtatgttttttagtgttttttaaagatttttattttttaatggaaatttagagttatttagtgattaaaagtaaaatgtttaaagtataaaatatgataataaaaatttttagtaaaagagaaaagtaattatttgatttcgggttttgatgtttagagataaaattgtatttagtttaggtttataaaagtagaatttttataaattaaattgattaagttttttgt

SEQ ID NO: 4 - DNA, 3581 bp, Artificial Sequence
  chemically treated genomic DNA (Homo sapiens)
ataaaagatttagttaatttaatttgtaagaattttatttttgtagatttgaattggatataattttatttttggatattaaagtttggggttaaatgattgttttttttttttgttgggagtttttgttgttatgttttatattttagatattttatttttggttattaggtggttttgaatttttattagaaagtgaaagtttttagaaaatattggaaaatatatataatttttattaaataattttaaaatattttatttaataatgattgatttgagaatattaattaaagtttattaaaagatttataaattttaaatagtattttgttaatttattttatgtagattttgagatagattatttatagttttatatgaattatagtttttgtattggttataggtataatttttaaatgatttataaattttgttttaaataatgttattttggagttatattatattatgtattatgtatggttaaataattgggtttgttatatttaaaagataattttaaaatattgttatataattttaatggttttaaagttttttttaagtgtattttagtagtttttaatgtatttggtgttaattttgaaattatttttaaggttagtaattttaaataaaaagaatggttagaataaggatttttagttagtttggaagttattaatagtttttttttgttgttggaaaaagtgatttttttgttttgagttttataataattattaaatgtgttgtatttgatttttgtaaagatttaatttttttttaaatttttaatgataatattttttgttattttattaaaaagtgtgttaatataggaagtagtgatttaaagttatttttggataatttattaataagggaggtaggaatggtggttgtagaaatgtgtgtagagagagtaattattatgggaataatttagatggtggtttgtttggtttgaattttttggaagaggtttatttttaaaatgtttaatattattttttattatagattttatatatgtttattttttaataaattttgagagaggtggtgagatatttttgtagtaattttatttttagttttttggattattttttttaaattttttaaattttttatgtttttttttatatgtttaaaatgtaggtagtgtagaatgattgtttgtggtttttttagttttagtttttgtatggaggtggaggtttttatatatatgattatatgagattgagttttagagttaggttttttgttatgagtatggttgtggatatgtaaggagttttagtggttttataatttggtaattttaatttaaataaattaggtaaaatgtttttagttggatttaagataaattttaggtagagtaatttttaaaagaaattagtaaattggatatttggtttttgatttataatttttttaaaatagataaaatagttttttttattttttattgtgtttttggttttaattttattgaatggttgttatttttgagatatattttttattgtaaatttgtattttttttattttgtgtattatttttttttttagttttatttggtaatgtgtatttttttttaagttttgtgttaaatgatgtgaagattgaggtttttttttggttggggtagtattatagtattatgataaaaagtttaggtgttgtttgtattggaagttaagttaattattggttatgagattttgagtaagttatttaattttagttgttatatttataaaatgggattatagtaggattattggtgttatagagttatgtgaggtttaaataagttattttaaaggatttagtatagtatttggtatattatgtggtttaagtattaataggttttgtagtaagtgtagttatttaagtgagttataataagtataggtgtgtttttgggagtgtttattttaagtttgaattagatgtaattattttttatttttttggagaaaatatatttgtaatattattttaaatataagttggtggtgtatttgtttagttttattaatgaattgggatgatataaaagtaatgaatagatgaagttttatattttttgtttttaagagaggatttggtgttgtttatttagtttttttgttttttttgtgttttttgaggattgagagattaaaagttatgttggtggatattgtagaagagtaggtggggaggagaatggtatggtagtgataggaagtgttgggaggtttaatatggagaaggagaggggtagtggggaagagggagtgggataaagggtatggggaagtatggggaagagggagtttttttttgtgtttagatagtgagaggtgtttatgagttaataaataaaagaggaaaggaaatttaatgagttgttatggattaagtattttgattgagttatattgtttgaaagatattagtaaagatgggaatttatatttggataagattttattagaggagtattttaggaattgatttgtggattttaattttgttagggttaaaagattatttgttgggtaagggtaggattaataattttatttataatgtatttattgatttgttgatttatagagtaaatatttttgaataatttttgtgtttttggtattgttttaggtattagtgatataggagttaataagatagatatgttattgtttttatggagttgtattttagtgtatggaggtagaaaataaataaataaataaataaataaataaataaataagataatttttaatagtaatgtgttaatatagtgtgatgggaaggagtatgatgagatagaaggaaggtttaaattgggaaatttgagaaatggtatggttgtatgtgggttggtatttttgtatgatgggagtggttatttgtttttatattttgaagttagagtgttttagatagaagaaatagtaagtgttgagaagttggtattagaaaaatagaggggagatttgtgtggttgtagttgagggagattaggaagatttgtatggtgggaaggatttgatgatatagaggtttgtaggttatgggaatgggtttggaattttatttaaagagttatgggaagtgattagaaggtttaaagttggggaagaggttttgtgtttttgttatatttgtgttttatataaattattttggttgttgtgttgataggatagtagaagggtaggagtagggatattagttaagttattgtaatggttaaggtgagaggtggtggttgggtttaggtttttttggagtgaataagttatttgtttggtttttatgatttttttttttttattggaatggaaggtatttgatattagagatttttgtttgttttattattattgtaattttaatgtttataatagattttggtatatagaagatatttgtttaatgttgttgattaattatattgagagagaagaatgatgtagagtagatttagagaaaaagttaaaggttttgtttagtatatgttaaatttgagatatttattagatatttaaatggagatatt

SEQ ID NO: 5 - DNA, 3581 bp, Artificial Sequence
  chemically treated genomic DNA (Homo sapiens)
gatatttttatttaaatatttaatgggtattttaaatttaatatgtgttaaatagaatttttgattttttttttaaatttgttttgtattattttttttttttagtatagttagttagtaatattaaataagtgttttttatgtgttagagtttgttgtagatattggggttatagtagtgatgaaatagataaaaatttttaatattaagtattttttattttagtggagggagaaggattatggaagttaagtaaatagtttgtttattttaaaaaggtttaggtttagttattattttttattttaattattgtaataatttaattggtgtttttgtttttatttttttgttgttttattaatatagtaattagagtaatttgtataaaatataaatataatagaaatataaaattttttttttaattttaaattttttagttattttttatagttttttgaataaaattttaaatttatttttatggtttatagatttttgtattattaggttttttttattatgtagatttttttggttttttttggttgtagttatataaattttttttttgtttttttgatgttagttttttggtatttgttattttttttgtttggaatattttgattttagaatatgaaagtaggtggttatttttattatgtaagaatgttaatttatatataattatattattttttagattttttagtttaaattttttttttgttttattatgtttttttttgttatattatgttgatatgttgttattaaaaattattttatttatttatttatttatttatttgtttgtttgttttttgtttttatgtattgaaatgtagttttatgagagtagtgatatgtttgttttgttggtttttatattattggtgtttagaatagtgttagaaatataggagttgtttagaagtatttgttttgtgaattaatgaattaatgaatgtattgtgaatgaggttattggttttatttttgtttaataaataattttttaattttaatgaagttgagatttataggttaatttttaaggtgtttttttagtgaagttttgtttaaatgtgagtttttatttttattaatattttttaaatagtgtaatttaattgaagtgtttagtttatggtaatttattaaattttttttttttttttatttattgatttatgggtattttttattgtttaaatatagaagaaagtttttttttttttatgtttttttatgttttttattttatttttttttttttattatttttttttttttttatattgggttttttagtattttttgttattgttatattatttttttttttatttgtttttttgtagtatttattaatgtggtttttaattttttagtttttagagagtataggagaggtaaggaagttaaatagatgatattagatttttttttggggatgggggatatgaaattttatttgtttattatttttgtgttattttagtttattggtagaattgggtaagtatattattagtttgtgtttgaagtggtattatgagtatgttttttttaggagagtagagagtagttgtgtttaatttagatttggggtgagtatttttagaagtatgtttatgtttgttatggtttatttagatgattatatttattgtaaagtttattaatatttaagttatatagtatattaggtattgtattaagttttttaaggtaatttatttaaattttatgtaattttgtgatattagtagttttgttatgattttattttatggatgtggtaattgaaattaagtaatttgtttaagattttatgattagtaattgatttgatttttagtgtaggtagtatttgggttttttgttatgatgttataatgttgttttgattagaagaagattttagtttttatattgtttgatataggatttgaggaaaagtatatattgttagatggagttagaaggggagatggtgtatgaggtgggaggagtgtaggtttgtggtagaggatgtgttttagaaatggtaattatttaatgaggttggagttaaagatatagtggggagtgaaggaagttattttgtttgttttaaaaaaattgtaaattagaaattaaatatttagtttgttggttttttttgaggattgttttgtttgggatttattttgagtttaattaaaaatgttttatttggtttgtttggattaagattgttaagttgtggagttattgaggttttttatgtgtttatagttatgtttatggtgaagggtttgattttggagtttaattttatgtggttgtgtatgtagggatttttgtttttatataagagttaggattggaggagttatagatagttattttatattgtttgtattttgaatatgtaaaaagaaatgtggagagtttggagagtttgaaaaggatggtttagggggttgggggtaaaattattgtaagagtattttattattttttttagagtttgttaagaaatgaatatgtgtggaatttatagtaaaaagtggtattaagtattttaaagatgggttttttttagaaagtttgagttaagtagattgttatttggattatttttatggtagttgttttttttgtgtatatttttgtaattgttatttttgttttttttattaataggttatttggaaatgattttggattattgttttttatgttaatatattttttgatgaaataatagagagtattgttattaaagatttggaaaaaagttaaatttttataggagttaaatgtagtatatttaatggttgttgtagaatttagggtaaagaaattattttttttaataatagaaggaaattgttggtgatttttaaattggttaaaggtttttgttttaattattttttttgtttaaaattgttaattttaaaaatgattttagagttggtattagatgtattggaggttgttaaagtatatttaggaaagattttaaagttattaaagttatatgatagtattttgaagttgttttttaaatatgatggatttagttgtttgattatatatgatatataatatgatatagttttagagtggtattatttggaatagagtttatgaattatttaaaaattgtatttataattaatgtaaaagttataatttatgtaaagttataaataatttattttaaaatttatataagatagattgatagagtgttatttggagtttatgggttttttgataaattttgattaatatttttaagttaattattgttgaatgaagtattttaaaattatttggtgaaaattatgtatgttttttagtgttttttaaagatttttattttttaatggaaatttagagttatttagtgattaaaagtaaaatgtttaaagtataaaatatgataataaaaatttttagtaaaagagaaaagtaattatttgattttgggttttgatgtttagagataaaattgtatttagtttaggtttataaaagtagaatttttataaattaaattgattaagttttttgt

SEQ ID NO: 6 - DNA, 18 bp, Artificial Sequence
  Hybridisation detection oligonucleotide
taaagttcggggttaaat

SEQ ID NO: 7 - DNA, 18 bp, Artificial Sequence
  Hybridisation detection oligonucleotide
taaagtttggggttaaat

SEQ ID NO: 8 - DNA, 18 bp, Artificial Sequence
  Hybridisation detection oligonucleotide
ttgggttcgttatattta

SEQ ID NO: 9 - DNA, 18 bp, Artificial Sequence
  Hybridisation detection oligonucleotide
ttgggtttgttatattta

SEQ ID NO: 10 - DNA, 18 bp, Artificial Sequence
  Hybridisation detection olignonucleotide
agttattttcggataatt

SEQ ID NO: 11 - DNA, 18 bp, Artificial Sequence
  Hybridisation detection olignonucleotide
agttatttttggataatt

SEQ ID NO: 12 - DNA, 18 bp, Artificial Sequence
  Hybridisation detection olignonucleotide
aatggcggttgtagaaat

SEQ ID NO: 13 - DNA, 18 bp, Artificial Sequence
  Hybridisation detection olignonucleotide
aatggtggttgtagaaat

SEQ ID NO: 14 - DNA, 18 bp, Artificial Sequence
  Hybridisation detection olignonucleotide
ttagatggcggtttgttt

SEQ ID NO: 15 - DNA, 18 bp, Artificial Sequence
  Hybridisation detection olignonucleotide
ttagatggtggtttgttt

SEQ ID NO: 16 - DNA, 18 bp, Artificial Sequence
  Hybridisation detection olignonucleotide
aattttttacgttttttt

SEQ ID NO: 17 - DNA, 18 bp, Artificial Sequence
  Hybridisation detection olignonucleotide
aattttttatgttttttt

SEQ ID NO: 18 - DNA, 18 bp, Artificial Sequence
  Hybridisation detection olignonucleotide
tatggaggcggaggtttt

SEQ ID NO: 19 - DNA, 18 bp, Artificial Sequence
  Hybridisation detection olignonucleotide
tatggaggtggaggtttt

SEQ ID NO: 20 - DNA, 18 bp, Artificial Sequence
  Hybridisation detection olignonucleotide
tatatacgattatatgag

SEQ ID NO: 21 - DNA, 18 bp, Artificial Sequence
  Hybridisation detection olignonucleotide
tatatatgattatatgag

SEQ ID NO: 22 - DNA, 18 bp, Artificial Sequence
  Hybridisation detection olignonucleotide
gtttttcgttatgagtat

SEQ ID NO: 23 - DNA, 18 bp, Artificial Sequence
  Hybridisation detection olignonucleotide
gttttttgttatgagtat

SEQ ID NO: 24 - DNA, 18 bp, Artificial Sequence
  Hybridisation detection olignonucleotide
tggatacgtaaggagttt

SEQ ID NO: 25 - DNA, 18 bp, Artificial Sequence
  Hybridisation detection olignonucleotide
tggatatgtaaggagttt

SEQ ID NO: 26 - DNA, 18 bp, Artificial Sequence
  Hybridisation detection olignonucleotide
aggtaaaacgtttttagt

SEQ ID NO: 27 - DNA, 18 bp, Artificial Sequence
  Hybridisation detection olignonucleotide
aggtaaaatgtttttagt

SEQ ID NO: 28 - DNA, 18 bp, Artificial Sequence
  Hybridisation detection olignonucleotide
tttttatcgtaaatttgt

SEQ ID NO: 29 - DNA, 18 bp, Artificial Sequence
  Hybridisation detection olignonucleotide
tttttattgtaaatttgt

SEQ ID NO: 30 - DNA, 18 bp, Artificial Sequence
  Hybridisation detection olignonucleotide
tttatttcgtgtattatt

SEQ ID NO: 31 - DNA, 18 bp, Artificial Sequence
  Hybridisation detection olignonucleotide
tttattttgtgtattatt

SEQ ID NO: 32 - DNA, 18 bp, Artificial Sequence
  Hybridisation detection olignonucleotide
tgtgttaaacgatgtgaa

SEQ ID NO: 33 - DNA, 18 bp, Artificial Sequence
  Hybridisation detection olignonucleotide
tgtgttaaatgatgtgaa

SEQ ID NO: 34 - DNA, 18 bp, Artificial Sequence
  Hybridisation detection olignonucleotide
tttttggtcggggtagta

SEQ ID NO: 35 - DNA, 18 bp, Artificial Sequence
  Hybridisation detection olignonucleotide
tttttggttggggtagta

SEQ ID NO: 36 - DNA, 18 bp, Artificial Sequence
  Hybridisation detection oligonucleotide
agagttacgtgaggttta

SEQ ID NO: 37 - DNA, 18 bp, Artificial Sequence
  Hybridisation detection oligonucleotide
agagttatgtgaggttta

SEQ ID NO: 38 - DNA, 18 bp, Artificial Sequence
  Hybridisation detection olignonucleotide
agtataggcgtgtttttg

SEQ ID NO: 39 - DNA, 18 bp, Artificial Sequence
  Hybridisation detection olignonucleotide
agtataggtgtgtttttg

SEQ ID NO: 40 - DNA, 18 bp, Artificial Sequence
  Hybridisation detection olignonucleotide
gaattagacgtaattatt

SEQ ID NO: 41 - DNA, 18 bp, Artificial Sequence
  Hybridisation detection olignonucleotide
gaattagatgtaattatt

SEQ ID NO: 42 - DNA, 18 bp, Artificial Sequence
  Hybridisation detection olignonucleotide
aatatattcgtaatatta

SEQ ID NO: 43 - DNA, 18 bp, Artificial Sequence
  Hybridisation detection olignonucleotide
aatatatttgtaatatta

SEQ ID NO: 44 - DNA, 18 bp, Artificial Sequence
  Hybridisation detection olignonucleotide
tatttttcgtttttaaga

SEQ ID NO: 45 - DNA, 18 bp, Artificial Sequence
  Hybridisation detection olignonucleotide
tattttttgtttttaaga

SEQ ID NO: 46 - DNA, 18 bp, Artificial Sequence
  Hybridisation detection olignonucleotide
ttggtgtcgtttatttag

SEQ ID NO: 47 - DNA, 18 bp, Artificial Sequence
  Hybridisation detection olignonucleotide
ttggtgttgtttatttag

SEQ ID NO: 48 - DNA, 18 bp, Artificial Sequence
  Hybridisation detection olignonucleotide
aaagttacgttggtggat

SEQ ID NO: 49 - DNA, 18 bp, Artificial Sequence
  Hybridisation detection olignonucleotide
aaagttatgttggtggat

SEQ ID NO: 50 - DNA, 18 bp, Artificial Sequence
  Hybridisation detection olignonucleotide
taagtatttcgattgagt

SEQ ID NO: 51 - DNA, 18 bp, Artificial Sequence
  Hybridisation detection olignonucleotide
taagtattttgattgagt

SEQ ID NO: 52 - DNA, 18 bp, Artificial Sequence
  Hybridisation detection olignonucleotide
tttaatttcgttagggtt

SEQ ID NO: 53 - DNA, 18 bp, Artificial Sequence
  Hybridisation detection olignonucleotide
tttaattttgttagggtt

SEQ ID NO: 54 - DNA, 18 bp, Artificial Sequence
  Hybridisation detection olignonucleotide
tattgattcgttgattta

SEQ ID NO: 55 - DNA, 18 bp, Artificial Sequence
  Hybridisation detection olignonucleotide
tattgatttgttgattta

SEQ ID NO: 56 - DNA, 18 bp, Artificial Sequence
  Hybridisation detection olignonucleotide
aatagtaacgtgttaata

SEQ ID NO: 57 - DNA, 18 bp, Artificial Sequence
  Hybridisation detection olignonucleotide
aatagtaatgtgttaata

SEQ ID NO: 58 - DNA, 18 bp, Artificial Sequence
  Hybridisation detection olignonucleotide
atagtgtgacgggaagga

SEQ ID NO: 59 - DNA, 18 bp, Artificial Sequence
  Hybridisation detection olignonucleotide
atagtgtgatgggaagga

SEQ ID NO: 60 - DNA, 18 bp, Artificial Sequence
  Hybridisation detection olignonucleotide
gttgtagtcgagggagat

SEQ ID NO: 61 - DNA, 18 bp, Artificial Sequence
  Hybridisation detection olignonucleotide
gttgtagttgagggagat

SEQ ID NO: 62 - DNA, 18 bp, Artificial Sequence
  Hybridisation detection olignonucleotide
gtaagtgtcgagaagttg

SEQ ID NO: 63 - DNA, 18 bp, Artificial Sequence
  Hybridisation detection olignonucleotide
gtaagtgttgagaagttg

SEQ ID NO: 64 - DNA, 18 bp, Artificial Sequence
  MS SnuPE detection oligonucleotide for DD3
tttttggatattaaagtt

SEQ ID NO: 65 - DNA, 18 bp, Artificial Sequence
  MS SnuPE detection oligonucleotide for DD3
tggttaaataattgggtt

SEQ ID NO: 66 - DNA, 18 bp, Artificial Sequence
  MS SnuPE detection oligonucleotide for DD3
gtgatttaaagttatttt

SEQ ID NO: 67 - DNA, 18 bp, Artificial Sequence
  MS SnuPE detection oligonucleotide for DD3
taagggaggtaggaatgg

SEQ ID NO: 68 - DNA, 18 bp, Artificial Sequence
  MS SnuPE detection oligonucleotide for DD3
tgggaataatttagatgg

SEQ ID NO: 69 - DNA, 18 bp, Artificial Sequence
  MS SnuPE detection oligonucleotide for DD3
aattttttaaatttttta

SEQ ID NO: 70 - DNA, 18 bp, Artificial Sequence
  MS SnuPE detection oligonucleotide for DD3
ttagtttttgtatggagg

SEQ ID NO: 71 - DNA, 16 bp, Artificial Sequence
  MS SnuPE detection oligonucleotide for DD3
gaggtttttatatata

SEQ ID NO: 72 - DNA, 18 bp, Artificial Sequence
  MS SnuPE detection oligonucleotide for DD3
ttttagagttaggttttt

SEQ ID NO: 73 - DNA, 18 bp, Artificial Sequence
  MS SnuPE detection oligonucleotide for DD3
tgagtatggttgtggata

SEQ ID NO: 74 - DNA, 18 bp, Artificial Sequence
  MS SnuPE detection oligonucleotide for DD3
taaataaattaggtaaaa

SEQ ID NO: 75 - DNA, 18 bp, Artificial Sequence
  MS SnuPE detection oligonucleotide for DD3
ttgagatatattttttat

SEQ ID NO: 76 - DNA, 18 bp, Artificial Sequence
  MS SnuPE detection oligonucleotide for DD3
tttgtattttttttattt

SEQ ID NO: 77 - DNA, 18 bp, Artificial Sequence
  MS SnuPE detection oligonucleotide for DD3
tttaagttttgtgttaaa

SEQ ID NO: 78 - DNA, 18 bp, Artificial Sequence
  MS SnuPE detection oligonucleotide for DD3
ttgaggtttttttttggt

SEQ ID NO: 79 - DNA, 18 bp, Artificial Sequence
  MS SnuPE detection oligonucleotide for DD3
attggtgttatagagtta

SEQ ID NO: 80 - DNA, 18 bp, Artificial Sequence
  MS SnuPE detection oligonucleotide for DD3
agttataataagtatagg

SEQ ID NO: 81 - DNA, 18 bp, Artificial Sequence
  MS SnuPE detection oligonucleotide for DD3
ttttaagtttgaattaga

SEQ ID NO: 82 - DNA, 18 bp, Artificial Sequence
  MS SnuPE detection oligonucleotide for DD3
ttttggagaaaatatatt

SEQ ID NO: 83 - DNA, 18 bp, Artificial Sequence
  MS SnuPE detection oligonucleotide for DD3
atgaagttttatattttt

SEQ ID NO: 84 - DNA, 18 bp, Artificial Sequence
  MS SnuPE detection oligonucleotide for DD3
taagagaggatttggtgt

SEQ ID NO: 85 - DNA, 18 bp, Artificial Sequence
  MS SnuPE detection oligonucleotide for DD3
ttgagagattaaaagtta

SEQ ID NO: 86 - DNA, 18 bp, Artificial Sequence
  MS SnuPE detection oligonucleotide for DD3
gttatggattaagtattt

SEQ ID NO: 87 - DNA, 18 bp, Artificial Sequence
  MS SnuPE detection oligonucleotide for DD3
atttgtggattttaattt

SEQ ID NO: 88 - DNA, 18 bp, Artificial Sequence
  MS SnuPE detection oligonucleotide for DD3
ataatgtatttattgatt

SEQ ID NO: 89 - DNA, 18 bp, Artificial Sequence
  MS SnuPE detection oligonucleotide for DD3
gataatttttaatagtaa

SEQ ID NO: 90 - DNA, 16 bp, Artificial Sequence
  MS SnuPE detection oligonucleotide for DD3
tgttaatatagtgtga

SEQ ID NO: 91 - DNA, 18 bp, Artificial Sequence
  MS SnuPE detection oligonucleotide for DD3
agaagaaatagtaagtgt

SEQ ID NO: 92 - DNA, 18 bp, Artificial Sequence
  MS SnuPE detection oligonucleotide for DD3
gatttgtgtggttgtagt

SEQ ID NO: 93 - DNA, 22 bp, Artificial Sequence
  primer example 1
tggttttaattttattgaatgg

SEQ ID NO: 94 - DNA, 22 bp, Artificial Sequence
  primer example 1
aaacaaatacaccaccaactta

SEQ ID NO: 95 - DNA, 18 bp, Artificial Sequence
  MS SNuPE probe example 1
attggtgttatagagtta

SEQ ID NO: 96 - DNA, 20 bp, Homo Sapiens
aagtgagccataacaagcat

SEQ ID NO: 97 - DNA, 20 bp, Homo Sapiens
cttttgtgtcatcccagttc